(19) United States
(12) Patent Application Publication
(10) Pub. No.: US 2005/0256594 A1
(43) Pub. Date: Nov. 17, 2005

(54) Digital noise filter system and related apparatus and methods

(76) Inventors: Sui-Kay Wong, North Point (CN); Kwok Yau (William) Cheung, Tai Po N.T. (CN); Wai Choi (Lewie) Leung, Chai Wan (CN)

Correspondence Address: Kevin J. McGough, 714 Colorado Avenue, Bridgeport, CT, 06605, US

(21) Appl. No.: 11/112,381

(22) Filed: Apr. 22, 2005

Related U.S. Application Data
(60) Provisional application No. 60/566,298, filed on Apr. 29, 2004.

(51) Int. Cl. G06F 17/00; A61F 11/06

(52) U.S. Cl. 700/94; 381/72; 381/94.1

(57) ABSTRACT

The invention provides a programmable, power-efficient, hearing aid and cell phone-compatible system for selectively filtering continuous mechanical noise which includes a programmable, user-adjustable DSP comprising a multiband equalizer, noise reduction and anti-feedback function, and advanced gain control (AGC) volume control.

### RELATED APPLICATIONS

This application claims priority from U.S. Provisional Patent Application No. 60/566,298, filed Apr. 29, 2004.

### FIELD OF THE INVENTION

The invention provides a programmable, power-efficient, hearing aid and cell phone-compatible system for selectively filtering continuous mechanical noise which includes a programmable, user-adjustable DSP comprising a multiband equalizer, noise reduction and anti-feedback function, and advanced gain control (AGC) volume control.

### BACKGROUND OF THE INVENTION

Hearing loss is generally associated with a loss of hearing sensitivity, which is a function of frequency. The most common type of sensitivity loss is an increasing function of frequency. Sensitivity is typically a function of speech level as well. Hence, loud sounds should be amplified less than soft sounds. It has been long known that a hearing aid should treat the various frequency components of speech differently to render them intelligible to a hearing impaired person.

In many cases the response of the ear of a hearing impaired person will be substantially different in terms of sensitivity and frequency response from that of a normal person. In a sensorineural hearing impairment, soft sounds are rendered inaudible, while loud sounds may be subjectively just as loud as for a normal person. Conversational levels may be very soft or even inaudible for a person with sensorineural hearing impairment. Consequently, if linear amplification is used to assist such a person, loudness relationships are perceived as distorted, and loud sounds may be uncomfortably loud. It is necessary in such cases to raise the amplitude of soft speech cues to the level of audibility. Beyond this, further improvements may be had by reestablishing loudness relationships. In many instances, a hearing impaired person will only experience a hearing loss at high frequencies and at low levels. For such a person it is desirable to provide a device, which amplifies sound only at low levels and high frequencies.

Individuals with impaired hearing often experience difficulty understanding conversational speech in background noise. The majority of daily conversations occur in background noise of one form or another. In some cases, the background noise may be more intense than the target speech, resulting in a severe signal-to-noise ratio problem.

Background mechanical noise (e.g., noise emitted by air conditioners, fans, air dryers, cars, trains, and machines) is usually continuous, with noise frequency and amplitude usually not varying by more than 5% or so. Such continuous mechanical noise therefore essentially reflects a spectrum of fixed amplitude sinusoidal signals that are subject to destructive interference when combined with a 180° off (half phase off) destructive signal. (Under such conditions, the “peak” of the fixed amplitude sinusoidal signal is in phase with the “valley” of the destructive wave and reduces the fixed amplitude sinusoidal signal to zero amplitude.)

In a conventional hearing aid, a microphone converts incident sound waves into an analog electrical signal, which is then processed to filter out unwanted noise, amplified, and coupled to a receiver or speaker which converts the electrical signal back to sound waves. The electrical signal processor may be an analog processor, which operates directly upon an analog electrical signal. Known analog hearing aids use relatively simple methods to alter their frequency shaping and dynamic range compression to mitigate the loss in hearing sensitivity for frequency and level. Alternatively, the analog signal may be converted to a digital signal and processed by a digital signal processor (DSP). Various hearing aids and related systems, including noise reduction systems, are described in U.S. Pat. Nos. 6,292,571; 6,236,731; 6,240,192; and 6,104,822.

General-purpose DSP chips provide an architecture that is flexible and that may be used in hearing aid-related systems. A generic DSP device consists of various functional blocks including accumulators, adder/subtractors, multipliers, registers, data memory, and program memory. These functional blocks are multiplexed to minimize the number of blocks needed. When two numbers need to be multiplied, the numbers are retrieved from memory and latched into the input registers of the multiplier. The output of the multiplier contains the result, which is then stored in another register or in memory.

The design of digital hearing aids involves numerous trade-offs between processing capability, flexibility, power consumption and size. Minimizing both chip size and power consumption are important design considerations for integrated circuits such as DSP's used in hearing aids. Fully programmable implementations of digital hearing aids (i.e., those that use a software-controlled digital signal processor) provide the most flexibility. However, with current technology, a fully programmable DSP chip or core consumes a relatively large amount of power. An application specific processor (typically implemented using an application specific integrated circuit or ASIC) will consume less power and chip-area than a fully programmable, general-purpose DSP core for equivalent processing capabilities, but is less flexible and adaptable.

Many hearing aid instruments have been designed to compress the dynamic range of the input sound signal so that it more nearly fits into the residual dynamic range of the recruited ear. The ratio of input dynamic range in dB to compressor output dynamic range in dB is called the compression ratio. To adequately specify the compressor, the compression ratio needs to be accompanied by a static gain value. This static gain value will determine at which input power level the system delivers a specified fixed gain. For example the static gain may be set so that at 80 dB SPL input power, the system delivers unity gain. If the compressor is set to a 2:1 compression ratio, then at 60 dB SPL input power the system will produce a 70 dB SPL output, that is a gain of 10 dB, and at 100 dB SPL input power the system will produce a 90 dB SPL output, that is a gain of −10 dB.

Usually the compression ratio is not constant over the entire input power range. A low-level compression knee may be defined. For input powers below this low-level compression knee, the compression ratio may be 1:1, that is, a fixed linear gain may be applied. The designated compression ratio (e.g. 2:1) may take affect only for input power levels above this low-level compression knee. A high level or limiting knee may also be defined. For input power levels above this high level knee, the compression ratio may increase or even become infinite, or it may be that the output level is fixed regardless of increase in input level. A system which has only a high-level compression knee below which the compression ratio is 1:1 (linear gain) is called a limiter. A system which has a low-level compression knee positioned at 40-50 dB SPL is termed a full range compressor.

The compression process requires a means for measuring the power of the input signal and generating a dynamically varying gain as a function of this input power. This gain is then applied to the signal which is delivered to the ear. When the input power is low, this gain will generally be high so that soft sounds are made louder. When the input power is high, this gain will generally be low so that loud sounds are not made too loud. The measure of input power requires averaging over time. The time span of the averaging defines a compression time constant. If the time span is very long then the compressor will react slowly to changes in input power level. This is sometimes referred to as Automatic Gain Control (AGC) where time constants of one to two seconds are typical. When the time span of the averaging is short the compressor will react quickly to changes in input power level. With a time span of approximately five to fifty milliseconds, the compressor may be referred to as a syllabic rate compressor. A syllabic rate compressor will limit the gain of a loud vowel sound while amplifying a soft consonant which immediately follows it.

In most designs there is both an attack and release compressor time constant. The attack time constant determines the time it takes for the compressor to react at the onset of a loud sound. That is, the time it takes to turn down the gain. The release time constant determines the time it takes for the system to turn up the gain again after the loud sound has terminated. Most often the attack time is quite short (<5 milliseconds) with the release time being longer (anywhere from 15 to 100 s of milliseconds).

To match the variability of recruitment with frequency, a compressor is often designed to perform differently in different frequency bands. A multi-band compressor divides the input signal into multiple frequency bands and then measures power in each band and compresses each band separately with possibly different compression ratios and time constants in the different bands. For example a properly designed two band compressor can make soft high frequency consonants audible while suppressing low frequency competing noises occurring simultaneously. The outer hair cells of the cochlea, when functioning normally, are often thought to perform compression function in overlapping frequency bands called critical bands. These frequency bands are spaced linearly at intervals of approximately 100 Hz at frequencies below about 500 Hz, and are spaced logarithmically at approximately third octave intervals above 500 Hz. Thus, the outer hair cells behave as a biological critical band compressor. The time constant associated with this compressor has been approximated to be about 1 ms.

As the number of compression bands increases, each with its own compression ratio and static gain, it is possible to view the compressor as having an almost continuously varying compression ratio as a function of frequency. In this case the system may, be represented as a set of frequency dependent gain curves. Each gain curve applies at a certain input power level. For input between these power levels, the system interpolates between gain curves.

The process of adjusting the compression ratios or gain curves of a compressor is central to the hearing aid fitting process. One approach to doing this is to attempt to adjust the compressor so that for all input levels and all frequencies the hearing impaired listener has the same impression of loudness that a normal listener would have. Loudness is a perceptual quantity which can under certain constraints be plotted as a function of input power level. The loudness growth curve may be measured by presenting a number of input signals at different levels and asking the listener to subjectively rate these on a perceptual scale (e.g. 1 to 10). By measuring the loudness growth curves of an impaired listener at different frequencies and comparing these to the loudness growth curves of an average of normal listeners, a loudness matching compression fitting can be attempted. To accurately match loudness growth curves, the hearing instrument would permit continuously variable compression ratio over input level. In this case it is more useful to think in terms of continuously variable input/output power curves. The system described above with low and high-level compression knees is able to implement only three segment piecewise input output curves.

The need still exists for a system that filters a broad range of continuous mechanical noise to facilitate the use of auditory-related apparatus such as hearing aids, auditory protection devices, and portable phones (e.g., cell phones). A power-efficient, cell phone-compatible system that could be employed in a hearing aid or auditory protection device to selectively filter continuous mechanical noise in accordance with a user's particular auditory profile would benefit not only to the hearing impaired, but also those of ordinary hearing that desire to filter out distracting or dangerous mechanical noise.

### SUMMARY OF THE INVENTION

The invention provides a power-efficient, hearing aid and cell phone-compatible system for selectively filtering continuous mechanical noise. Systems of the invention include a programmable, user-adjustable DSP comprising a multiband equalizer, noise reduction and anti-feedback function and advanced gain control (AGC) volume control. Systems of the invention can be used, e.g., in hearing aids; in auditory protective devices used to protect against the dangers, discomforts, and inconveniences of continuous mechanical noise; and with cell phones to filter unwanted background noise.

In one embodiment, systems and methods of the invention facilitate cell phone usage by serving as a noise-reducing ear-piece component that does not create unwanted interference. In another embodiment, systems and methods of the invention are used in combined hearing aid-cell phone applications.

Systems of the invention may be calibrated through a novel calibration routine by programming the DSP multiband equalizer to: select one or more of at least around twenty-three different bandwidths based on a user's auditory profile; and filter continuous mechanical noise.

Significantly, systems and methods of the invention enable a user to self-calibrate, either manually or through interconnection with a PC program, a multiband equalizer in accordance with the user's particular auditory frequency responses. Further, the DSP used in the invention does not interfere with the electromagnetic impulse of portable phones such as cellular phones.

An auditory protection system of the invention comprises:
a signal adder that adds a plurality of audio input signals to generate a DSP input signal, wherein at least one of the audio input signals is representative of a sound level associated with continuous mechanical noise; and
a DSP which processes in discrete time increments the DSP input signal to filter continuous mechanical noise. The DSP comprises a noise reduction and anti-feedback function, a multiband equalizer with a variable compression ratio and bandwidth, and an advanced gain control (AGC) volume control.

In a preferred embodiment: the DSP is programmed through use of a personal computer to provide a frequency response that is optimized for a particular user and that is selected from at least around twenty-three bandwidths ranging from 50 Hz to 8,000 Hz. In preferred embodiments, the DSP noise reduction function generates, in intervals of between about 1 to about 7 seconds, signals having a frequency of about 50 Hz to about 8,000 Hz and amplitudes of about 70 dB to about 130 dB. These signals achieve substantially complete destructive interference of continuous mechanical noise in the range of 0 to about 15 dB. In a preferred embodiment, the DSP output signal is defined by a frequency response of around 2,000 Hz to around 2,500 Hz; and the volume of the DSP output signal is about 130 dB.

The invention also provides systems and methods for calibration of a system for processing digital signals in a hearing aid and/or auditory protection device. Such calibration comprises:
activation of an appropriate display and selection and activation from the display of one or more parameters including an indicator of a directional microphone, an indicator of an omni directional microphone, an indicator for selection of mobile phone usage, and an indicator for calibration lock;
adjustment of speaker volume;
optional receipt or transmission by an interconnected portable phone of incoming or outgoing phone calls;
retrieval and implementation of a system menu by optionally unlocking a calibration function, selecting a display language, selecting audio input signal microphones, selecting noise reduction options correlated to a percentage of a dB rating of continuous mechanical noise;
interconnection with a personal computer for implementation of a self-calibration routine including multiband equalizer band selection in accordance with instructions provided by a program operating on the personal computer; and
selection and adjustment of multiband equalizer through selection of one of several available bandwidths until an appropriate band is determined.

A hearing aid that employs systems of the invention (collectively, the “unit”) offers numerous advantages over, and differs from, traditional BTH (behind the ear)/ITH (in the ear)/ITC (in the canal) type hearing aids. For example, the unit battery and PCBA compartment may be separated from the microphone and or earphone; the unit can use a battery that is rechargeable; the unit is connectable to a mobile phone and may be used with a hands-free microphone and earphone set while the input signal is processed through the unit and heard by the user; the unit's multiband equalizer can be adjusted by the user either by a built-in user self-calibration function or through a PC link program; mechanical noise reduction strength can be selected by the user according to the environment; and the unit itself will be styled like a fashion electronic appliance such as MP3 or mobile phone, contributing to a completely different appearance than that of a traditional hearing aid.

Further advantages offered by the instant invention include the following:
1. The invention reduces mechanical noise over several different levels of noise. Mechanical noise reduction levels can be adjusted by the user
2. The invention achieves multi-frequency band equalizing that divides the hearing frequency bandwidths, from about 50 Hz to about 8,000 Hz, into at least around twenty-three bandwidths. The amplification of each bandwidth can be adjusted individually.
3. The invention provides an anti-feedback function.
4. The invention provides an auditory advanced gain control (AGC) function.
5. The multiband equalizer of the invention can be adjusted by the user either by a built-in user self-calibration function or through a PC-device linked program.
6. The built-in user self-calibration function of the invention will generate frequency signals sufficient to allow to a user to hear and adjust amplification to an appropriate comfort level.
7. Systems of the invention are cell phone-compatible and function as a hands-free device comprising all above mentioned features of multi-frequency band equalization, filtering of continuous mechanical noise, anti-feedback, and AGC.

These and other aspects of the invention are described in further detail in the following detailed description.

### BRIEF DESCRIPTION OF THE DRAWINGS

FIG. 1A illustrates a block diagram of one embodiment of a system of the instant invention.

FIG. 1B illustrates the physical connection of one embodiment of a system of the instant invention.

FIG. 2 illustrates a flowchart depicting one embodiment of a calibration routine that may be employed for user calibration of the instant invention.

### DETAILED DESCRIPTION OF THE INVENTION

The embodiments of the invention described herein are purely illustrative. Other embodiments of the invention will be recognized by those of ordinary skill in the art.

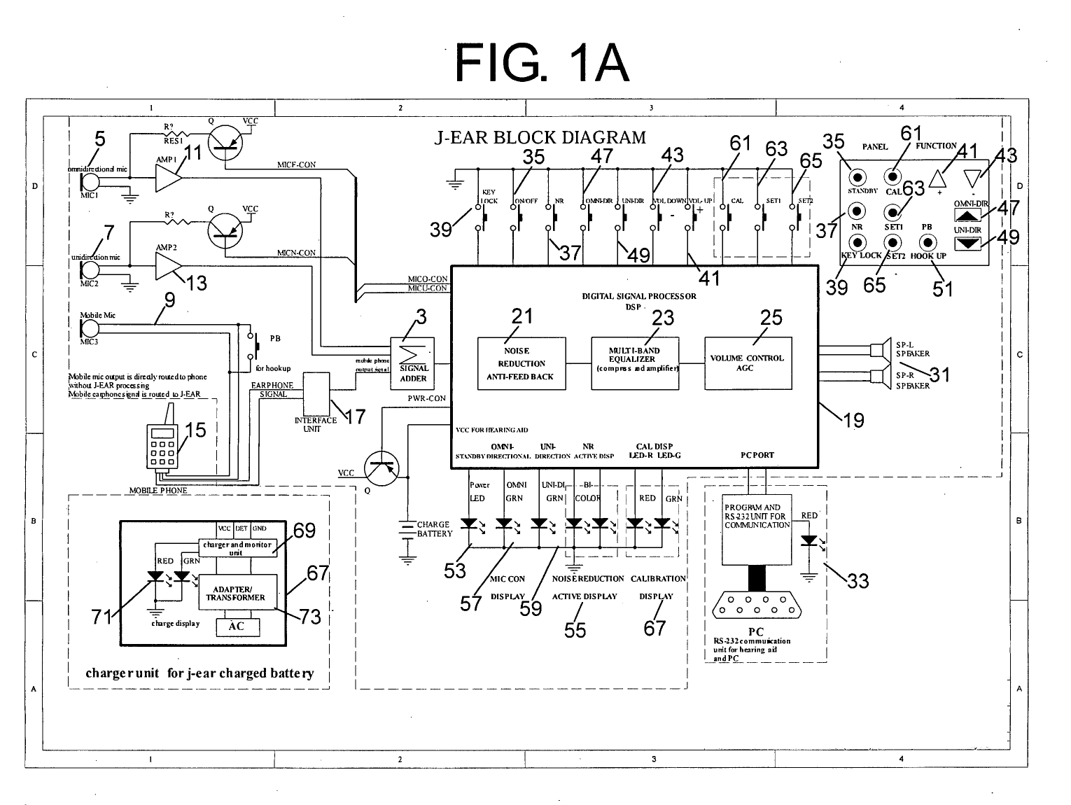

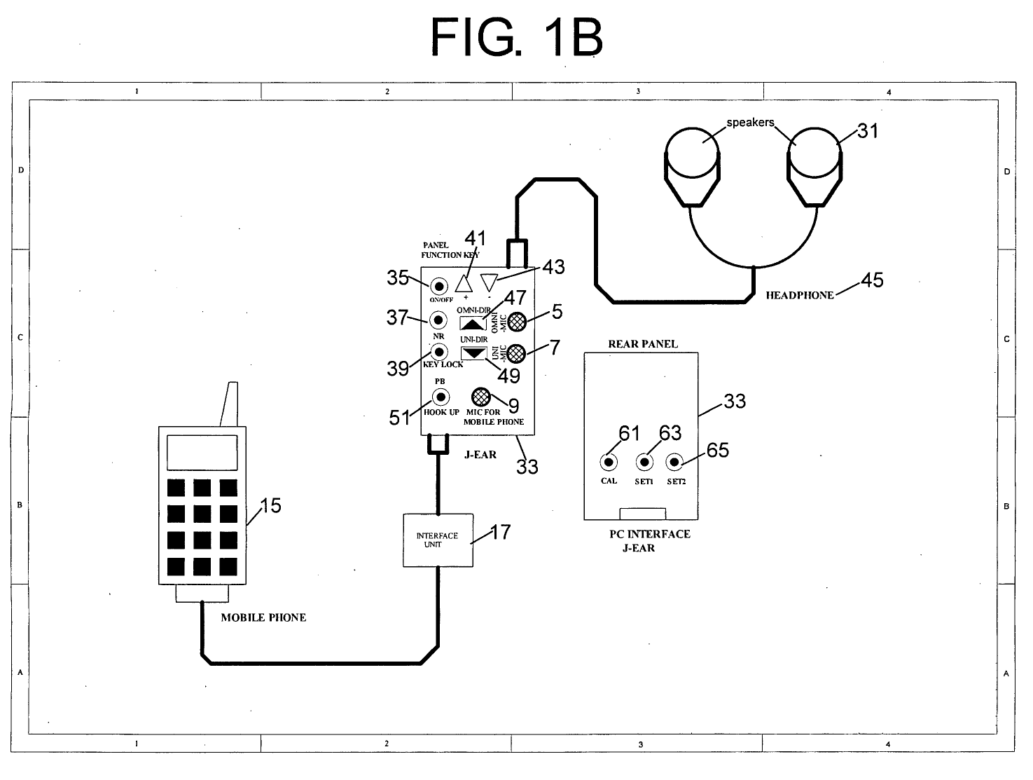

FIGS. 1A and 1B illustrate an embodiment of the invention wherein a system for processing digital signals in a hearing aid or auditory protection device is used in conjunction with a portable phone (e.g., a cell phone), and in which a DSP is programmed through use of a personal computer to provide a frequency response that is optimized for a particular user. In FIG. 1A, signal adder 3 adds input signals: received by omni-directional microphone 5 and amplified by amplifier 11; received by unidirectional microphone 7 and amplified by amplifier 13; and received by mobile phone microphone 9 and routed through mobile phone 15 and interface unit 17. The signal adder 3 output signal constitutes the input signal to DSP 19. DSP 19 comprises noise reduction and anti-feedback function 21, multiband equalizer 23 having a variable compression ratio of approximately from about 4:1 to about 1:1 and a bandwidth of approximately from about 50 Hz to about 8,000 Hz, and AGC volume control 25.

Feedback is generated when amplified sound from hearing aid speakers 31 leaks back to the microphones 5, 7, and 9 and is re-amplified. The anti-feedback function 21 separates the signal which leaks from the hearing aid amplifier and normal input sound and then eliminates the leakage sound. AGC volume control 25 limits output volume to a specific dB level.

DSP 19 can comprise a single integrated circuit (e.g., integrated circuits SSCM 2167 (Analog Devices), Paragon® Digital filter (Gennum), or Paragon® Digital GB3215 (Gennum)), that has been programmed to include a noise reduction and anti-feedback function 21, multiband equalizer 23 with a variable compression ratio of approximately from about 4:1 to about 1:1 and a bandwidth of approximately from about 50 Hz to 8,000 Hz, and AGC volume control 25. In a preferred embodiment, DSP 19 is an integrated circuit that is programmed by interconnection to personal computer 33 or in accordance with a calibration routine for user adjustment of the multiband equalizer 23. The input signal to DSP 19 and the output signal from DSP 19 are transmitted to speakers 31. DSP 19 output signal transmitted to speakers 31 is defined by a frequency response at approximately 2,000 Hz; and the volume of the DSP 19 output signal is 130 dB.

FIGS. 1A and 1B illustrate the physical connection of the invention. The front panel of main unit 33 comprises on/off button 35 which controls the power on and off of the main unit 33. Power LED 53 illuminates when the power of main unit 33 is on. Noise reduction button 37 activates and/or deactivates the noise reduction function 21. Noise reduction LEDs 55 illuminate to show that the noise reduction function 21 is active. Key lock button 39 locks and unlocks all the functional keys onto the main unit 33. Volume up 41 and volume down 43 keys control the volume output through headphone 45 which contains speakers 31. Omni-directional 47 and unidirectional 49 buttons activate or deactivate the omni-directional 5 and unidirectional microphone 7 respectively. Omni-directional LED 57 and unidirectional LED 59 illuminate to show omni-directional microphone 5 and unidirectional microphone 7 are active respectively. A user receives an incoming mobile phone call by pressing the PB hook up key 51 and the incoming signal via mobile phone 15 and interface unit 17 to main unit 33. A signal from mobile phone microphone 9 is routed to mobile phone 15 via main unit 33 and interface unit 17. The rear panel of main unit 33 comprises user calibration button 61, set1 button 63 and set2 button 65. These three buttons, together with two calibration displays 67, control and display the user calibration routine in order to adjust the multiband equalizer 23 by the user. The charging unit illustrated in FIG. 1A comprises a circuit board 69 and a set of charging displays 71 by which AC-DC adaptor 73 is inserted to the charging unit 67 for power transmission.

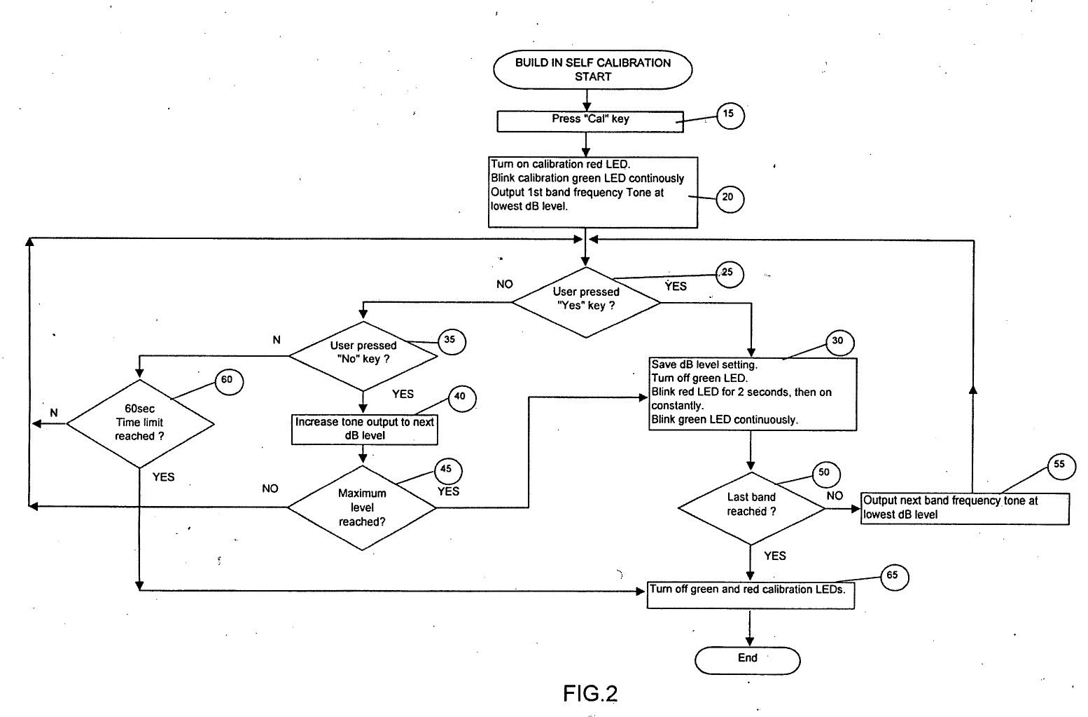

Calibration of DSP 19 in accordance with one embodiment of the instant invention is illustrated in FIG. 2. In the illustrated user calibration routine, the user presses the calibration key (e.g., FIG. 1A, element 61) to start 15 the calibration routine. DSP 19 generates 20 a single tone signal to the user through speakers (e.g., FIG. 1A, element 31) and then waits 25 for a response. If the user can hear a single tone signal, a positive response 63 will feedback 30 to DSP 19 and DSP 19 continues to generate the next bandwidth of single tone signal. If the user cannot hear a single tone signal, a negative response 65 will feedback 35 to DSP 19 and DSP 19 increases the db level 40 and again generates 45 a signal to the user again. If there is no response feedback to the program in around a one minute limit 60, a calibration display (e.g., FIG. 1A, element 67) turns off and the calibration routine will be terminated 65. DSP 19 generates the next bandwidth of a single tone signal when it received a positive response 63 from the user or the db level reaches the maximum value 50. This user calibration routine applies to at least twenty-three bandwidths of frequency 55 and bandwidths of approximately from about 50 Hz to about 8,000 Hz. The calibration display 67 turns off to show that the calibration routine is completed 65.

1. An auditory protection system for processing digital signals comprising:
a signal adder that adds a plurality of audio input signals to generate a DSP input signal, wherein at least one of the audio input signals is representative of a sound level associated with continuous mechanical noise; and
a DSP which processes in discrete time increments the DSP input signal to filter continuous mechanical noise, the DSP comprising a noise reduction and anti-feedback function, a multiband equalizer with a variable compression ratio and bandwidth, and an advanced gain control (AGC) volume control.

2. A system of claim 1, wherein the DSP is programmed through use of a personal computer to provide an optimized frequency response.

3. A system of claim 2, wherein the frequency response is selected from greater than about twenty-three bandwidths ranging from about 50 Hz to about 8,000 Hz.

4. A system of claim 1, wherein the DSP noise reduction function generates, in intervals of between about 1 to about 7 seconds, signals having a frequency of about 50 Hz to about 8,000 Hz and an amplitude of about 70 dB to about 130 dB.

5. A system of claim 1, wherein the system achieves substantially complete destructive interference of continuous mechanical noise in the range of 0 to about 15 dB.

6. A system of claim 5, wherein the DSP output signal is defined by a frequency response of about 2,000 Hz to about 2,500 Hz, and wherein the volume of the DSP output signal is about 130 dB.

7. A system of claim 1, wherein the system is incorporated in a hearing aid.

8. A system of claim 1, wherein the system is incorporated in an auditory protection device.

9. A system of claim 1, wherein the system is incorporated in a cell phone.

10. A system of claim 1, wherein the system is adapted for use in, and may be incorporated into, a combination of a hearing aid and a cell phone.

11. A method of calibrating a system of claim 1, comprising:
activating an appropriate display, and selecting and activating from the display of one or more parameters including an indicator of a directional microphone, an indicator of an omni-directional microphone, an indicator for selection of mobile phone usage, and an indicator for calibration lock;
adjusting the volume of a speaker interconnected with the system;
optionally receiving or sending phone calls by a cell phone interconnected with the system;
retrieving and implementing a system menu by optionally unlocking a calibration function, selecting a display language, selecting audio input signal microphones, and selecting noise reduction options correlated to a percentage of a dB rating of continuous mechanical noise;
interconnecting the system with a personal computer for implementation of a self-calibration routine including multiband equalizer band selection in accordance with instructions provided by a program operating on the personal computer; and
selecting and adjusting the multiband equalizer and frequency response through selection of one of several available bandwiths until an appropriate frequency is determined.

12. A method of claim 11, wherein the frequency response is selected from greater than about twenty-three bandwidths ranging from about 50 Hz to about 8,000 Hz.

13. A method of claim 11, wherein the DSP noise reduction function generates, in intervals of between about 1 to about 7 seconds, signals having a frequency of about 50 Hz to about 8,000 Hz and an amplitude of about 70 dB to about 130 dB.

14. A method of claim 11, wherein the system achieves substantially complete destructive interference of continuous mechanical noise in the range of 0 to about 15 dB.

15. A method of claim 11, wherein the DSP output signal is defined by a frequency response of about 2,000 Hz to about 2,500 Hz, and wherein the volume of the DSP output signal is about 130 dB.

16. A method of claim 11, wherein the system is incorporated in a hearing aid.

17. A method of claim 11, wherein the system is incorporated in an auditory protection device.

18. A method of claim 11, wherein the system is incorporated in a cell phone.

19. A method of claim 11, wherein the system is adapted for use in, and may be incorporated into, a combination of a hearing aid and a cell phone.

20. A method of claim 11, wherein a cell phone receives or sends one or more calls.
